(19) United States
(12) Patent Application Publication
(10) Pub. No.: US 2024/0366909 A1
(43) Pub. Date: Nov. 7, 2024

(54) Therapeutic Pillowcase with a Sleep-Aid System

(71) Applicant: Selena Porter, Las Vegas, NV (US)

(72) Inventor: Selena Porter, Las Vegas, NV (US)

(21) Appl. No.: 18/656,553

(22) Filed: May 6, 2024

Related U.S. Application Data
(60) Provisional application No. 63/500,200, filed on May 4, 2023.

(51) Int. Cl. A61M 21/00

(52) U.S. Cl. CPC A61M 21/00; A61M 2021/0016; A61M 2021/0022; A61M 2021/0027; A61M 2021/0066

(57) ABSTRACT

A therapeutic pillowcase with a sleep-aid system is an apparatus that enables the user to have a better sleep experience. The apparatus is designed to securely and comfortably retain several therapeutic devices that help the user to fall asleep as well as to stay asleep. To do so, the apparatus includes a pillowcase body, an eye-masking sleeve, and a plurality of therapeutic inserts. The pillowcase body corresponds to the structure of the apparatus designed to cover a pillow. The pillowcase body also retains the plurality of therapeutic inserts in a secure and comfortable manner. The eye-masking sleeve helps cover the user's eyes while the user is sleeping for a more comfortable sleeping experience. The plurality of therapeutic inserts include several therapeutic devices that provide different therapeutic treatments to the user. Each of the plurality of therapeutic inserts can be attached to the pillowcase body as desired.

The current application claims a priority to the U.S. provisional patent application Ser. No. 63/500,200 filed on May 4, 2023. The current application is filed on May 6, 2024, while May 4, 2024 was on a weekend.

### FIELD OF THE INVENTION

The present invention relates to bedding accessories and therapeutic systems. More specifically, the present invention discloses a pillowcase with therapeutic features that help the user to have better sleep.

### BACKGROUND OF THE INVENTION

According to the CDC, 32% of adults suffer from insomnia. During the recent pandemic, that number jumped to 50%. In a recent study, 39% of moms get less than five hours of sleep a night. Further, the Sleep Foundation says 65% of women do not get the recommended 7 to 9 hours of sleep nightly, and 30% of adults overall suffer from insomnia. When attempting to sleep or to rest, a person may need extra help to fall asleep or fall back to sleep. There are many available methods to prevent this and the most common method is to use sleeping pills or supplements. However, these solutions may not offer optimal sleep cycles for the user. There are other sleep-aid solutions available to help the user sleep better. For example, therapeutic accessories such as heating pads and aromatherapy devices are available that the user can wear while sleeping. However, these therapeutic devices can be uncomfortable to wear while sleeping, and wearing several separate devices while sleeping can be cumbersome to the user. Therefore, a device that can comfortably retain therapeutic accessories to soothe or comfort the user and/or assist the user in falling asleep is necessary.

An objective of the present invention is to provide a therapeutic pillowcase with a sleep-aid system. The therapeutic pillowcase of the present invention provides users with means to retain one or more therapeutic devices in a comfortable manner that aid the user sleep better. Another objective of the present invention is to provide a therapeutic pillowcase with a sleep-aid system that position the retained therapeutic devices in such a way that the user can receive the therapeutic benefits from each device. The present invention allows the user to selectively position a desired therapeutic device close to the target body area for treatment. Another objective of the present invention is to provide a therapeutic pillowcase with a sleep-aid system that can be easily cleaned and maintained. Additional features and benefits of the present invention are further discussed in the sections below.

### SUMMARY OF THE INVENTION

The present invention discloses a therapeutic pillowcase with a sleep-aid system. The present invention discloses a therapeutic pillowcase with means to hold different therapeutic devices in such a way that each therapeutic device is positioned against the target body area for treatment. The present invention preferably includes several storage pockets integrated throughout the therapeutic pillowcase to hold the desired therapeutic device. For example, a storage pocket positioned adjacent to the user's forehead can be designed to hold a cold pack to cool the user's forehead to decrease the brain temperature and reduce frontal cerebral metabolism, which helps promote sleep in insomnia patients. This storage pocket may cover the user's face, eyes, nose, and/or ears. Further, other storage pockets can be provided on different areas throughout the therapeutic pillowcase to retain other therapeutic devices that provide different therapeutical benefits to other body areas. For example, a secondary pillow can be inserted to provide support for the user's neck, cervical back, and shoulders of the user. Other therapeutical devices can include, but are not limited to, thermotherapy devices, massage therapy devices, audio therapy devices, etc. In addition, each storage pocket can include a closure flap that keeps the corresponding therapeutic device inside the storage pocket. Furthermore, other optional therapeutic devices such as soft fur, pom-poms, or squeeze balls can be removably attached to the therapeutic pillowcase to be used as pacifiers to help the user fall asleep.

### BRIEF DESCRIPTION OF THE DRAWINGS

FIG. 1 is a top-front-left perspective view of the present invention.

FIG. 2 is a bottom-front-right perspective view of the present invention.

FIG. 3 is a top-front-left perspective view of the pillowcase body of the present invention.

FIG. 4 is a bottom-front-left perspective view of the pillowcase body of the present invention.

FIG. 5 is a left view of the pillowcase body of the present invention.

FIG. 6 is a bottom view of the pillowcase body of the present invention.

FIG. 7 is a front view of the pillowcase body of the present invention.

FIG. 8 is a cross-sectional view taken along line 8-8 in FIG. 7.

FIG. 9 is a top-front-left perspective view of the eye sleeve of the present invention.

FIG. 10 is a bottom-rear-right perspective view of the eye sleeve of the present invention.

FIG. 11 is a top view of the eye sleeve of the present invention.

FIG. 12 is a left view of the eye sleeve of the present invention.

FIG. 13 is a front view of the eye sleeve of the present invention.

FIG. 14 is a cross-sectional view taken along line 14-14 in FIG. 13.

### DETAILED DESCRIPTION OF THE INVENTION

All illustrations of the drawings are for the purpose of describing selected versions of the present invention and are not intended to limit the scope of the present invention.

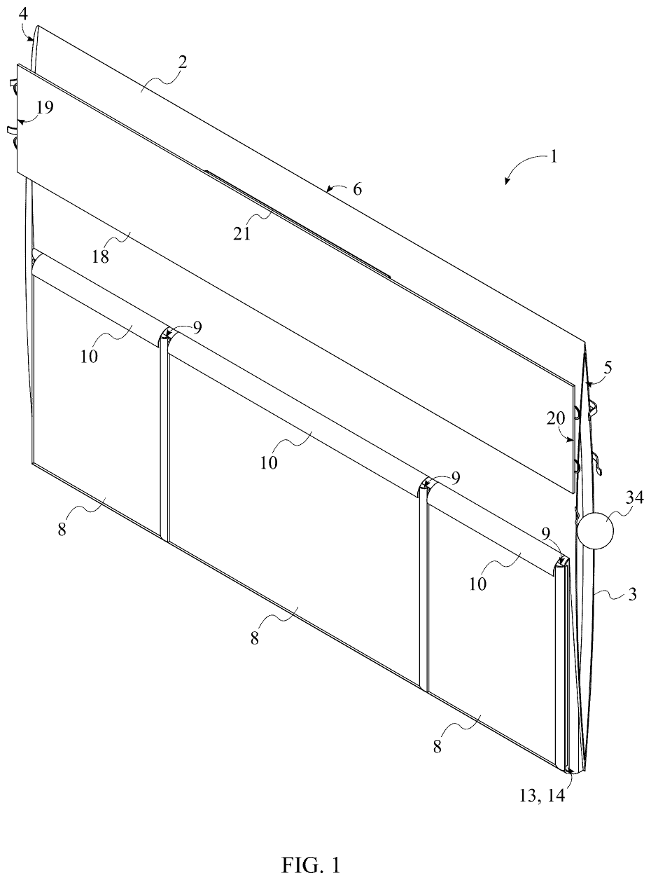

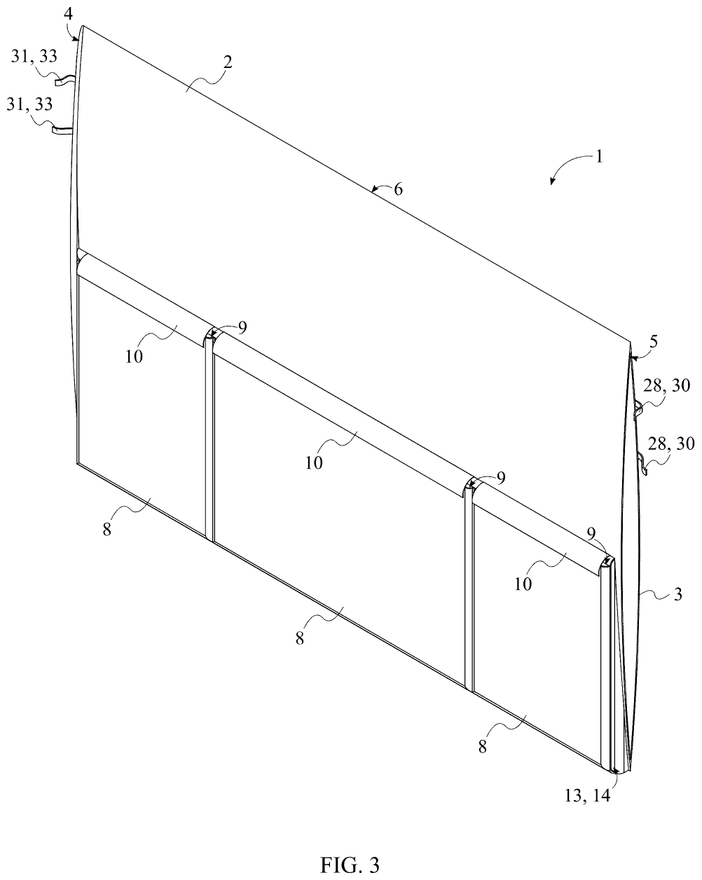

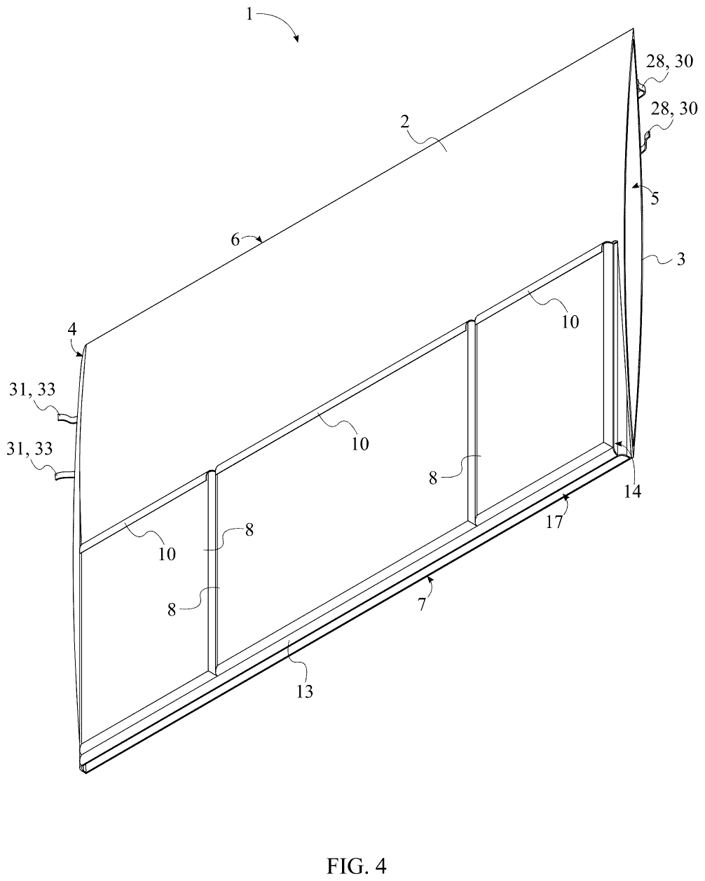

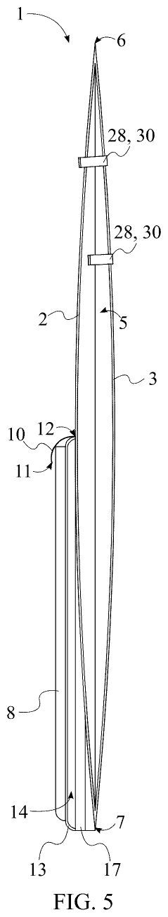

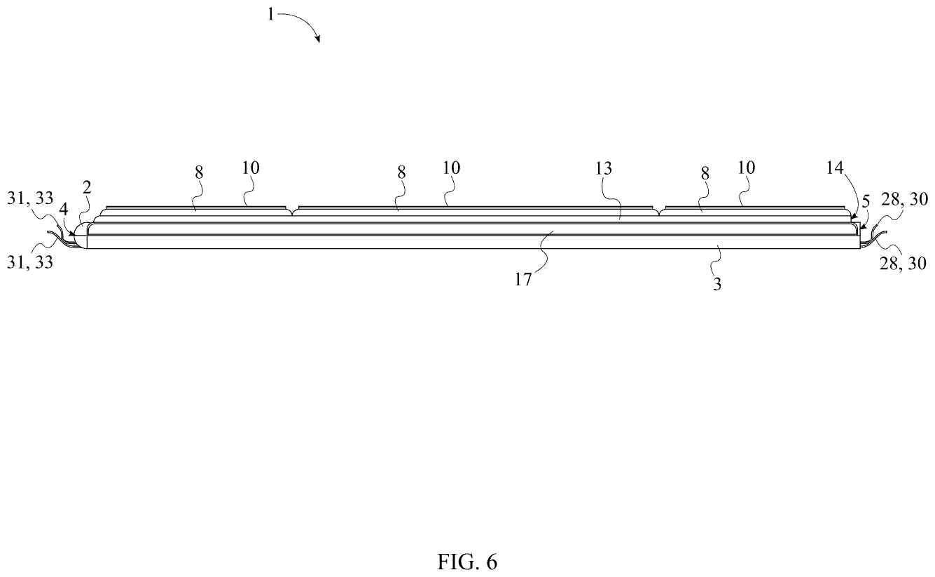

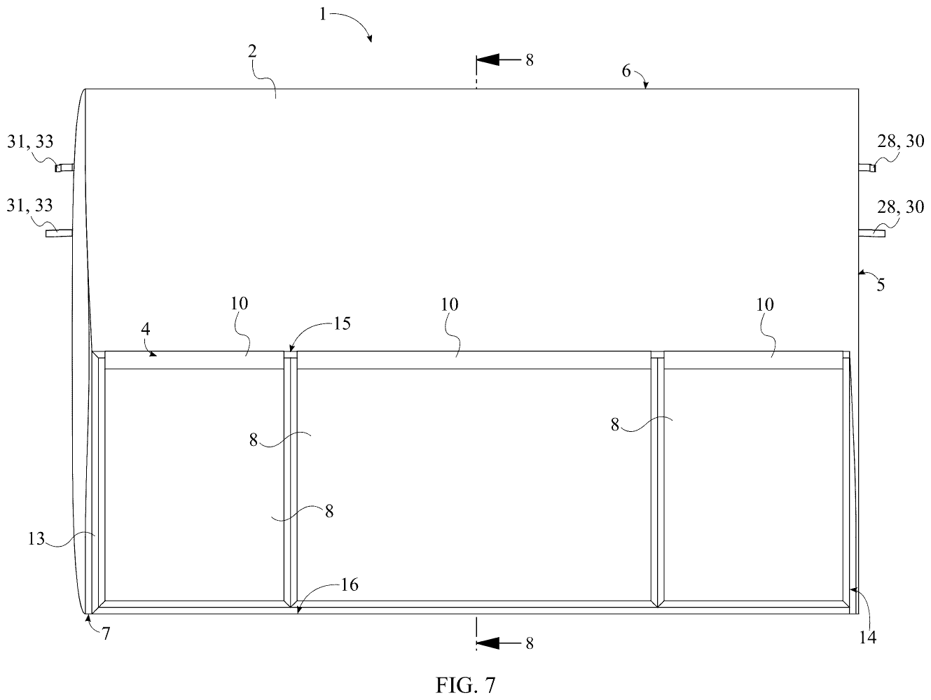

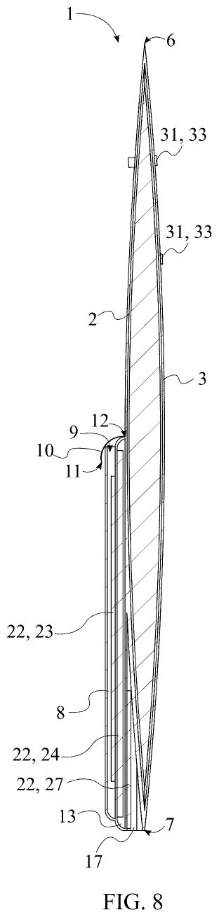

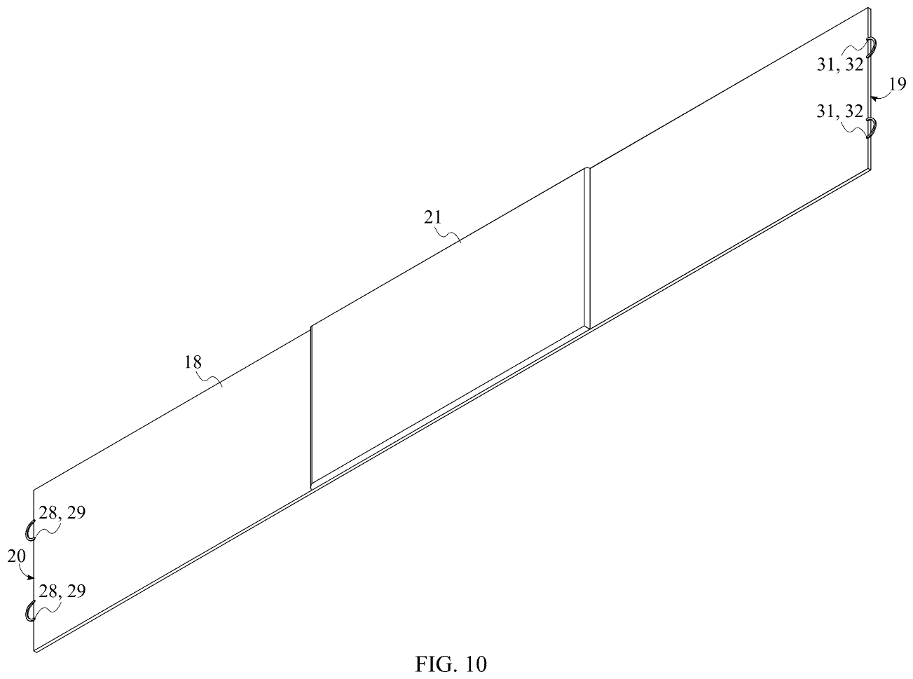

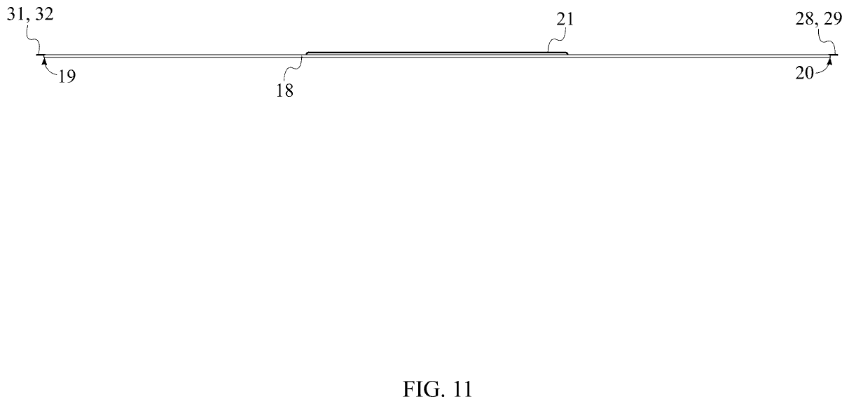

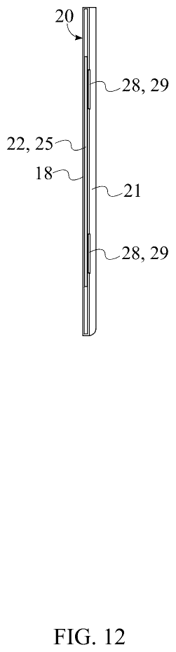

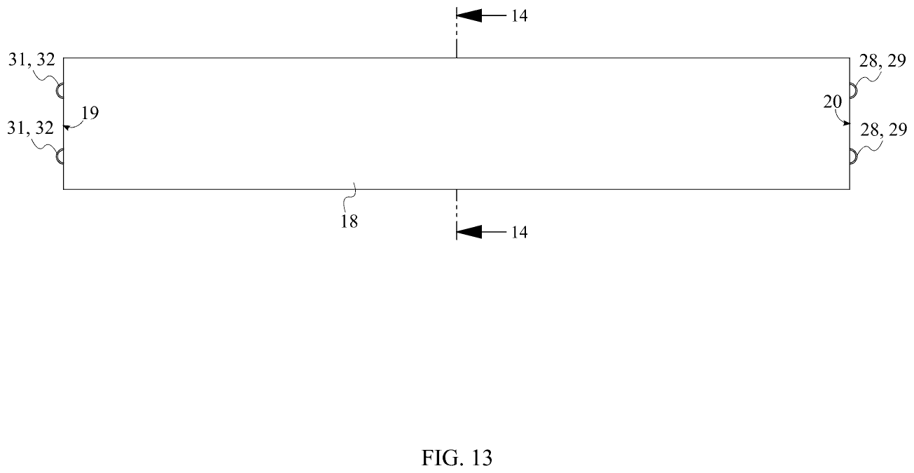

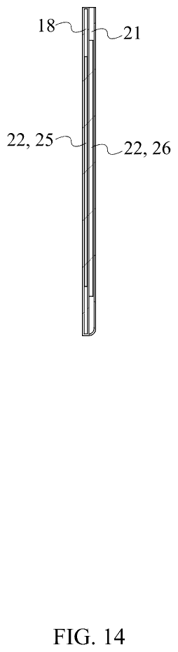

The present invention discloses a therapeutic pillowcase with a sleep-aid system that enables the user to have a better sleep experience. The present invention is designed to securely and comfortably retain several therapeutic devices to help the user sleep better. As can be seen in FIGS. 1 through 14, the present invention comprises a pillowcase body 1, eye-masking sleeve 18, and a plurality of therapeutic inserts 22. The pillowcase body 1 corresponds to the structure of the present invention designed to cover a pillow. The pillowcase body 1 also retains the plurality of therapeutic inserts 22 in a secure and comfortable manner external to the pillow. The eye-masking sleeve 18 helps cover the user's upper face while the user is sleeping for a more comfortable sleeping experience. The plurality of therapeutic inserts 22 include several devices that provide different therapeutic benefits to the user.

The general configuration of the aforementioned components enables the user to sleep better without taking medical supplements. As can be seen in FIGS. 1 through 8, the pillowcase body 1 is designed to cover the target pillow while also retaining the plurality of therapeutic inserts 22 in a safe and comfortable manner. The pillowcase body 1 is preferably designed to match the shape and size of the target pillow. For example, the pillowcase body 1 can have a rectangular design with a size large enough to tightly accommodate the pillow. However, the pillowcase body 1 can have other designs matching non-rectangular pillows or other bedding accessories. So, in general, the pillowcase body 1 comprises a front panel 2, a rear panel 3, a closed widthwise body edge 4, an open widthwise body edge 5, a first lengthwise body edge 6, a second lengthwise body edge 7, a plurality of short pockets 8, and an elongated pillow pocket 13. The front panel 2 and the rear panel 3 corresponds to two matching panels of the same size that are connected to each other to form a hollow structure that serves as the pillow's cover. The closed widthwise body edge 4, the first lengthwise body edge 6, and the second lengthwise body edge 7 correspond to the edges along which the front panel 2 and the rear panel 3 are connected to each other. The open widthwise body edge 5 corresponds to the edge of the pillowcase body 1 that is left unconnected to serve as the opening to the pillowcase body 1 through which the target pillow can be inserted into the pillowcase body 1. The plurality of short pockets 8 and the elongated pillow pocket 13 serve as storage structures integrated throughout the pillowcase body 1 designed to retain specific therapeutic inserts.

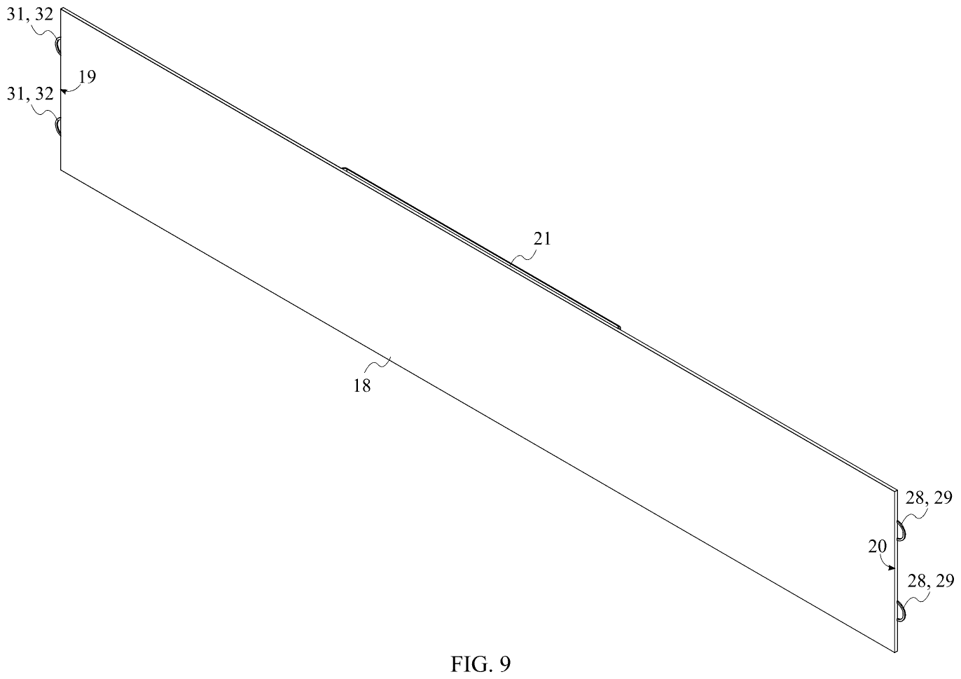

As can be seen in FIGS. 9 through 14, the eye-masking sleeve 18 is further designed to cover the user's upper face and/or middle face sections. For example, the eye-masking sleeve 18 can be an elongated structure made from several layers of material with an overall rectangular design large enough to cover the user's eyes, ears, and/or forehead. The eye-masking sleeve 18 can be made from elastic material so that the eye-masking sleeve 18 rests tightly against the user's face. Further, the eye-masking sleeve 18 is designed to retain one or more therapeutic inserts of the plurality of therapeutic inserts 22 to provide comfort and therapeutical benefits to the user. So, the eye-masking sleeve 18 comprises a closed sleeve end 19 and an open sleeve end 20 corresponding to the terminal ends of the eye-masking sleeve 18. In other embodiments, the pillowcase body 1 and/or the eye-masking sleeve 18 can be modified to accommodate different therapeutic accessories to help the user sleep.

As can be seen in FIGS. 1 through 14, in the preferred embodiment, the present invention can be arranged as follows: The eye-masking sleeve 18 is positioned adjacent to front panel 2, opposite to the rear panel 3, to cover the user's upper face when the user is resting on the pillow covered by the pillowcase body 1. In addition, the closed sleeve end 19 is peripherally mounted to the rear panel 3, adjacent to the closed widthwise body edge 4, to secure the closed sleeve end 19 to the pillowcase body 1. Similarly, the open sleeve end 20 is peripherally attached to the rear panel 3, adjacent to the open widthwise body edge 5, to secure the open sleeve end 20 to the pillowcase body 1. Further, the elongated pillow pocket 13 is integrated onto the front panel 2, opposite to the rear panel 3, to incorporate the elongated pillow pocket 13 on the pillowcase body 1. In addition, the eye-masking sleeve 18 and the elongated pillow pocket 13 are positioned offset to each other across the front panel 2 to accommodate the user's face anatomy. In other words, the eye-masking sleeve 18 is positioned to match the user's upper face, while the elongated pillow pocket 13 is positioned to match the user's neck. Further, the plurality of short pockets 8 is integrated into and along the elongated pillow pocket 13, opposite to the front panel 2, so that each of the plurality of short pockets 8 are positioned against the user's neck. This way, several of the plurality of therapeutic inserts 22 can be inserted into the different storage structures throughout the pillowcase body 1 and the eye-masking sleeve 18. For example, a first therapeutic insert 23 of the plurality of therapeutic inserts 22 can be positioned within a selected short pocket of the plurality of short pockets 8. A second therapeutic insert 24 of the plurality of therapeutic inserts 22 can be positioned within the elongated pillow pocket 13. Furthermore, a third therapeutic insert 25 of the plurality of therapeutic inserts 22 can be positioned within the eye-masking sleeve 18. In other embodiments, different storage options can be provided on the pillowcase body 1 to retain additional therapeutic devices.

As can be seen in FIGS. 1 through 14, in some embodiments, the eye-masking sleeve 18 can be designed so that the closed sleeve end 19 is permanently secured to the pillowcase body 1 while the open sleeve end 20 can be selectively attached to the other side of the pillowcase body 1. In this embodiment, the present invention may further comprise a first sleeve fastening mechanism 28 that allows the open sleeve end 20 to be selectively attached to the pillowcase body 1. The first sleeve fastening mechanism 28 includes several fasteners with separate interlocking pieces that allow the open sleeve end 20 to be removably attached to the pillowcase body 1. So, the first sleeve fastening mechanism 28 comprises a plurality of first loops 29 and a plurality of first snap tabs 30. The plurality of first loops 29 and the plurality of first snap tabs 30 can be selectively engaged to each other to secure the open sleeve end 20 to the pillowcase body 1. To do so, the plurality of first loops 29 is distributed along the open sleeve end 20 to receive the corresponding first snap tabs of the plurality of first snap tabs 30. In addition, the plurality of first loops 29 is connected onto the open sleeve end 20 to secure each of the plurality of first loops 29 to the eye-masking sleeve 18. On the other hand, the plurality of first snap tabs 30 is connected onto the rear panel 3, adjacent to the open widthwise body edge 5 to secure each of the plurality of first snap tabs 30 to the rear panel 3. The distribution of the plurality of first snap tabs 30 preferably match the distribution of the plurality of first loops 29 to facilitate the attachment of the open sleeve end 20 to the rear panel 3. Furthermore, each first snap tab of the plurality of first snap tabs 30 is engaged with a corresponding first loop of the plurality of first loops 29 to secure the open sleeve end 20 to the pillowcase body 1.

As can be seen in FIGS. 1 through 14, in another embodiment, the eye-masking sleeve 18 can be designed so that the closed sleeve end 19 can also be removably attached to the pillowcase body 1. In this embodiment, the present invention may further comprise a second sleeve fastening mechanism 31 that allows the closed sleeve end 19 to be selectively attached to the pillowcase body 1. The second sleeve fastening mechanism 31 also includes several fasteners with separate interlocking pieces that allow the closed sleeve end 19 to be removably attached to the pillowcase body 1 as well. So, the second sleeve fastening mechanism 31 comprises a plurality of second loops 32 and a plurality of second snap tabs 33. The plurality of second loops 32 and the plurality of second snap tabs 33 can also be selectively engaged to each other to secure the closed sleeve end 19 to the pillowcase body 1. To do so, the plurality of second loops 32 is distributed along the closed sleeve end 19 to receive the corresponding second snap tabs of the plurality of second snap tabs 33. In addition, the plurality of second loops 32 is connected onto the closed sleeve end 19 to secure each of the plurality of second loops 32 to the eye-masking sleeve 18. In addition, the plurality of second snap tabs 33 is connected onto the rear panel 3, adjacent to the closed widthwise body edge 4, to secure each of the plurality of second snap tabs 33 to the rear panel 3. The distribution of the plurality of second snap tabs 33 preferably match the distribution of the plurality of second loops 32 to facilitate the attachment of the closed sleeve end 19 to the rear panel 3. Furthermore, each second snap tab of the plurality of second snap tabs 33 can be engaged with a corresponding second loop of the plurality of second loops 32 to secure the closed sleeve end 19 to the pillowcase body 1. In other embodiments, different sleeve fastening mechanisms can be implemented to help the user removably attach the eye-masking sleeve 18 to the pillowcase body 1.

In some embodiments, the eye-masking sleeve 18 is designed to retain one or more therapeutic inserts so that different therapeutical treatments can be applied to the user's middle face and/or upper face. As can be seen in FIGS. 9 through 14, the eye-masking sleeve 18 may further comprise at least one sleeve pocket 21 that retains an additional therapeutic insert separate from the third therapeutic insert 25 positioned inside the eye-masking sleeve 18. The at least one sleeve pocket 21 is centered along the eye-masking sleeve 18 to position the retained therapeutic insert on the center of the user's face. In addition, the at least one sleeve pocket 21 is oriented towards the first lengthwise body edge 6 to prevent the therapeutic insert from falling off. Further, a fourth therapeutic insert 26 of the plurality of therapeutic inserts 22 is positioned within the at least one sleeve pocket 21 to provide therapeutical treatment to the user's forehead. As previously discussed, the therapeutic inserts includes different therapeutic devices that provide different therapeutic treatments. For example, the fourth therapeutic insert 26 can be a thermotherapy insert such as a hot and/or cold pack that helps warm or cool the user's forehead. The fourth therapeutic insert 26 can also be an aromatherapy insert that helps calm the user down. In addition, the third therapeutic insert 25 can be a weighted sleeve insert to help the user have a good sleep. In other embodiments, different storage spaces can be provided on the eye-masking sleeve 18.

As can be seen in FIGS. 1 through 8, the elongated pillow pocket 13 preferably comprises a lateral pocket opening 14 that allows the second therapeutic insert 24 to be inserted into the elongated pillow pocket 13. In the preferred embodiment, the lateral pocket opening 14 is positioned adjacent to the open widthwise body edge 5 to allow the second therapeutic insert 24 to be inserted into the elongated pillow pocket 13 from the same side of the pillowcase body 1 as the pillow. Further, the second therapeutic insert 24 is preferably a therapeutic insert that provides additional support to the user's neck. For example, the second therapeutic insert 24 can be a neck pillow that can be inserted into the elongated pillow pocket 13 to provide support to the user's neck when the user is resting on the pillow inside the pillowcase body 1. In other embodiments, the lateral pocket opening 14 can be positioned on different locations on the pillowcase body 1 to allow the second therapeutic insert 24 to be inserted into the elongated pillow pocket 13 from different sides of the pillowcase body 1.

As can be seen in FIGS. 1 through 8, like the elongated pillow pocket 13, each of the plurality of short pockets 8 includes a corresponding opening to allow the insertion of the selected therapeutic insert into the corresponding short pocket. So, each of the plurality of short pockets 8 may comprise a front opening 9 and a closure flap 10. The front opening 9 allows the insertion of the selected therapeutic insert into the corresponding short pocket. In addition, the closure flap 10 keeps the selected therapeutic insert inside the corresponding short pocket by sealing the front opening 9. Further, each closure flap 10 comprises a free flap edge 11 and a fixed flap edge 12 corresponding to the terminal edges of each closure flap 10. The fixed flap edge 12 preferably corresponds to the edge of the closure flap 10 that is connected onto the pillowcase body 1, while the free flap edge 11 corresponds to the edge of the closure flap 10 that is left unconnected to the pillowcase body 1. In addition, the elongated pillow pocket 13 may further comprise a first lengthwise pocket edge 15 and a second lengthwise pocket edge 16 corresponding to the longer edges of the elongated pillow pocket 13.

As can be seen in FIGS. 1 through 8, in the preferred embodiment, the plurality of short pockets 8 can be arranged as follows: The elongated pillow pocket 13 is preferably long enough to match the length of the pillowcase body 1. So, the second lengthwise pocket edge 16 is positioned coincident with the second lengthwise body edge 7. Further, each front opening 9 is positioned adjacent to the first lengthwise pocket edge 15 to prevent the corresponding therapeutic insert from falling off the selected short pocket. In addition, the closure flap 10 is positioned adjacent to the front opening 9 so that each closure flap 10 can seal the corresponding front opening 9. Furthermore, the fixed flap edge 12 is connected onto the front panel 2, adjacent to the first lengthwise pocket edge 15, to secure the closure flap 10 to the pillowcase body 1 while leaving the free flap edge 11 loose to allow access into the selected short pocket through the corresponding front opening 9. In other embodiments, different sealing mechanisms can be used to selectively seal the front opening 9 of each of the plurality of short pockets 8 while enabling the corresponding first therapeutic insert 23 into the selected short pockets. As previously discussed, the plurality of therapeutic inserts 22 can include different types of therapeutic devices that provide different therapies. For example, the first therapeutic insert 23 can be an audio therapy insert that plays soothing or calming sounds to the user such as an audio media player. The first therapeutic insert 23 can also be a thermotherapy insert that warms or cools down the user's head. The first therapeutic insert 23 can further be a massage therapy insert that massages the user's face head while the user is sleeping. In other embodiments, different therapeutic inserts can be provided that can be inserted into the selected short pocket to apply different therapeutical treatments.

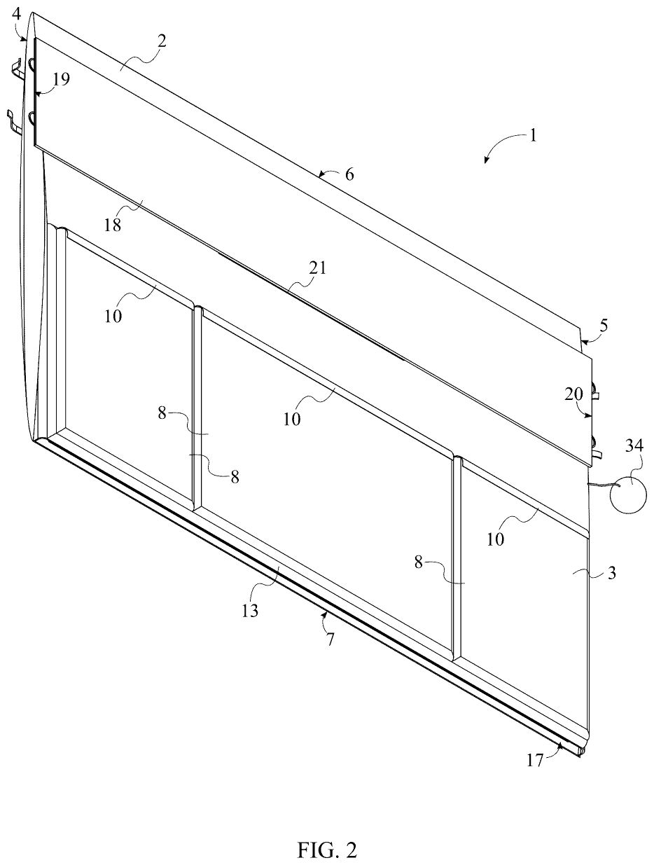

As can be seen in FIGS. 1 and 2, in some embodiments, the present invention can allow for secondary therapeutic devices to be removably attached to the pillowcase body 1. For example, the present invention may further comprise a stress-relief device 34 that the user can engage while trying to fall asleep. For example, the stress-relief device 34 can be a squeeze ball, a pom-pom ball, or other similar squeezable device that can be used to relief stress while the user is trying to fall asleep. The stress-relief device 34 is mounted onto the pillowcase body 1 so that the stress-relief device 34 can be engaged by the user while keeping the stress-relief device 34 attached to the pillowcase body 1. In other embodiments, different therapeutic devices can be provided that can be removably attached to the pillowcase body 1.

As can be seen in FIGS. 1 through 8, in some embodiments, the pillowcase body 1 can include additional storage spaces that allow for additional therapeutic devices to be retained on the pillowcase body 1. For example, the pillowcase body 1 may further comprise at least one body pocket 17 that retains an additional therapeutic insert that can is positioned adjacent to the pillow covered by the pillowcase body 1. The at least one body pocket 17 is preferably centered along the second lengthwise body edge 7 to center the corresponding therapeutic insert on the user's head. Further, a fifth therapeutic insert 27 of the plurality of therapeutic inserts 22 can be positioned within the at least one body pocket 17. For example, the fifth therapeutic insert 27 can also be a thermotherapy insert that helps warm up or cool down the pillow covered by the pillowcase body 1. In other embodiments, different therapeutic inserts can be positioned within the at least one body pocket 17. Additionally, other storage spaces can be provided throughout the pillowcase body 1.

In some embodiments, the present invention may further comprise a pillowcase fastener 35 that allows the pillowcase body 1 to be sealed to retain the pillow within the pillowcase body 1. The pillowcase fastener 35 can prevent accidental slippage of the pillowcase body 1 from the covered pillow while the user is resting on the pillow. So, the pillowcase fastener 35 is integrated along the open widthwise body edge 5, which allows the user to selectively seal the open widthwise body edge 5. For example, the pillowcase fastener 35 can be a zipper that can be selectively closed to seal the open widthwise body edge 5 and opened to have access to the inside of the pillowcase body 1. In other embodiments, different fasteners can be utilized to help seal the open widthwise body edge 5 as desired.

Although the invention has been explained in relation to its preferred embodiment, it is to be understood that many other possible modifications and variations can be made without departing from the spirit and scope of the invention.

What is claimed is:

1. A therapeutic pillowcase with a sleep-aid system comprising:
a pillowcase body;
an eye-masking sleeve;
a plurality of therapeutic inserts;
the pillowcase body comprising a front panel, a rear panel, a closed widthwise body edge, an open widthwise body edge, a first lengthwise body edge, a second lengthwise body edge, a plurality of short pockets, and elongated pillow pocket;
the eye-masking sleeve comprising a closed sleeve end and an open sleeve end;
the eye-masking sleeve being positioned adjacent to front panel, opposite to the rear panel;
the closed sleeve end being peripherally mounted to the rear panel, adjacent to the closed widthwise body edge;
the open sleeve end being peripherally attached to the rear panel, adjacent to the open widthwise body edge;
the elongated pillow pocket being integrated onto the front panel, opposite to the rear panel;
the eye-masking sleeve and the elongated pillow pocket being positioned offset to each other across the front panel;
the plurality of short pockets being integrated into and along the elongated pillow pocket, opposite to the front panel;
a first therapeutic insert of the plurality of therapeutic inserts being positioned within a selected pocket of the plurality of short pockets;
a second therapeutic insert of the plurality of therapeutic inserts being positioned within the elongated pillow pocket; and
a third therapeutic insert of the plurality of therapeutic inserts being positioned within the eye-masking sleeve.

2. The therapeutic pillowcase with a sleep-aid system as claimed in claim 1 comprising:
a first sleeve fastening mechanism;
the first sleeve fastening mechanism comprising a plurality of first loops and a plurality of first snap tabs;
the plurality of first loops being distributed along the open sleeve end;
the plurality of first loops being connected onto the open sleeve end;
the plurality of first snap tabs being connected onto the rear panel, adjacent to the open widthwise body edge; and
each of the plurality of first snap tabs being engaged with a corresponding first loop of the plurality of first loops.

3. The therapeutic pillowcase with a sleep-aid system as claimed in claim 1 comprising:
a second sleeve fastening mechanism;
the second sleeve fastening mechanism comprising a plurality of second loops and a plurality of second snap tabs;
the plurality of second loops being distributed along the closed sleeve end;
the plurality of second loops being connected onto the closed sleeve end;
the plurality of second snap tabs being connected onto the rear panel, adjacent to the closed widthwise body edge; and
each of the plurality of second snap tabs being engaged with a corresponding second loop of the plurality of second loops.

4. The therapeutic pillowcase with a sleep-aid system as claimed in claim 1 comprising:
the eye-masking sleeve further comprising at least one sleeve pocket;
the at least one sleeve pocket being centered along the eye-masking sleeve;
the at least one sleeve pocket being oriented towards to the first lengthwise body edge; and
a fourth therapeutic insert of the plurality of therapeutic inserts being positioned within the at least one sleeve pocket.

5. The therapeutic pillowcase with a sleep-aid system as claimed in claim 4, wherein the fourth therapeutic insert is a thermotherapy insert.

6. The therapeutic pillowcase with a sleep-aid system as claimed in claim 4, wherein the fourth therapeutic insert is an aromatherapy insert.

7. The therapeutic pillowcase with a sleep-aid system as claimed in claim 1, wherein the third therapeutic insert is a weighted sleeve insert.

8. The therapeutic pillowcase with a sleep-aid system as claimed in claim 1 comprising:
the elongated pillow pocket comprising a lateral pocket opening; and
the lateral pocket opening being positioned adjacent to the open widthwise body edge.

9. The therapeutic pillowcase with a sleep-aid system as claimed in claim 1, wherein the second therapeutic insert is a neck pillow.

10. The therapeutic pillowcase with a sleep-aid system as claimed in claim 1 comprising:
each of the plurality of short pockets comprising a front opening and a closure flap;
the elongated pillow pocket comprising a first lengthwise pocket edge and a second lengthwise pocket edge;
the closure flap comprising a free flap edge and a fixed flap edge;
the second lengthwise pocket edge being positioned coincident with the second lengthwise body edge;
the front opening being positioned adjacent to the first lengthwise pocket edge;
the closure flap being positioned adjacent to the front opening; and
the fixed flap edge being connected onto the front panel, adjacent to the first lengthwise pocket edge.

11. The therapeutic pillowcase with a sleep-aid system as claimed in claim 1, wherein the first therapeutic insert is an audio therapy insert.

12. The therapeutic pillowcase with a sleep-aid system as claimed in claim 1, wherein the first therapeutic insert is a thermotherapy insert.

13. The therapeutic pillowcase with a sleep-aid system as claimed in claim 1, wherein the first therapeutic insert is a massage therapy insert.

14. The therapeutic pillowcase with a sleep-aid system as claimed in claim 1 comprising:
a stress-relief device; and
the stress-relief device being mounted onto the pillowcase body.

15. The therapeutic pillowcase with a sleep-aid system as claimed in claim 1 comprising:
the pillowcase body further comprising at least one body pocket;
the at least one body pocket being centered along the second lengthwise body edge; and
a fifth therapeutic insert of the plurality of therapeutic inserts being positioned within the at least one body pocket.

16. The therapeutic pillowcase with a sleep-aid system as claimed in claim 15, wherein the fifth therapeutic insert is a thermotherapy insert.
